(19) United States
(12) Patent Application Publication
(10) Pub. No.: US 2025/0049989 A1
(43) Pub. Date: Feb. 13, 2025

(54) Tunnel Filler Device And Methods Of Use

(71) Applicant: Reprise Biomedical, Inc., Plymouth, MN (US)

(72) Inventors: Jason Isenburg, Victoria, MN (US); Kyle Bettinger, Blaine, MN (US)

(73) Assignee: Reprise Biomedical, Inc., Plymouth, MN (US)

(21) Appl. No.: 18/720,800

(22) PCT Filed: May 31, 2023
(86) PCT No.: PCT/US2023/067672

§ 371 Date: Jun. 17, 2024

Related U.S. Application Data
(60) Provisional application No. 63/347,362, filed on May 31, 2022.

(51) Int. Cl. A61L 27/36; A61B 17/00

(52) U.S. Cl. CPC A61L 27/3633; A61B 2017/00641; A61B 2017/00898

(57) ABSTRACT

Devices and methods for treatment of a tunnel wound in a mammal are disclosed. A device can include a suture, a tunnel filler, and a cap member. The suture can be folded to form a proximal loop and two distal ends. The tunnel filler can be compressed onto the suture between the proximal loop and the two distal ends. And the cap member can be positioned on the suture distal to the tunnel filler. The tunnel filler can comprise decellularized mammalian extracellular matrix. The device can further include a tamp member positioned over the suture distal to the cap member. A method of treating a tunnel wound can include introducing the device into the tunnel wound, applying pressure to an end of the tunnel filler by sliding the tamp member along the suture, removing the tamp member from the suture, and securing an end of the suture to an end of the tunnel wound.

### CLAIM OF PRIORITY

Benefit of priority is hereby claimed to U.S. Provisional Patent Application Ser. No. 63/347,362, titled “FISTULA DEVICE AND METHODS OF USE” and filed on May 31, 2022, which is herein incorporated by reference in its entirety.

### BACKGROUND

Fistulas are a major cause of morbidity and mortality. They cost the healthcare system billions of dollars each year to treat. Fistulas are tissue-lined connections between body cavities and hollow organs or between such cavities or organs and the surface of the body. A fistula tract includes a void in the soft tissues extending from a primary fistula opening to a blind ending or leading to one or more secondary fistula openings. Fistulas frequently develop because of infections or accompany abscess formations.

Fistulas can form between almost any two organ systems. For example, they may occur between internal organs and skin, such as enterocutaneous fistulas, gastrocutaneous fistulas, anal fistulas, rectovaginal fistulas, colocutaneous fistulas, vesiclocutaneous fistulas, intestinocutanous fistulas, tracheocutaneous fistulas, bronchocutaneous fistulas, etc., or between internal organs themselves, such as tracheal-esophogeal fistulas, gastrointestinal fistulas, colovesicular fistulas, palatal fistulas, etc. Fistulas may also form between blood vessels, such as arterial-venous fistulas.

Current options for treatment of fistulas include long-term conservative management or major surgery. Other treatment options may include implantable devices designed to aid in the closure of the fistula. These devices, however, may cause adverse immunological reactions in patients, may allow leakage of fluid around the device, or the device may migrate or become dislodged when the patient exerts himself/herself, such as during exercise.

An anal fistula, otherwise known as an anorectal fistula, is an abnormal passage formed between the wall of the anal canal and the skin around the anus, typically, the perianal skin. An anal fistula usually originates from an infection in an anal gland located in the anal canal. In the case of an anal gland becoming infected, an abscess may form deep under the skin around the anus which requires surgical drainage. After drainage, a tunnel (also referred to as a “tract” herein) between the drainage site and the wall of the anal canal may form resulting in an anal fistula. Fistulas cause intermittent symptoms of discharge and generally do not heal without treatment or surgical intervention. Anal fistulas are also a common feature of inflammatory bowel diseases, especially ulcerative colitis and Crohn's disease.

“Lay open” fistulotomy is the conventional surgery for treating an anal fistula and involves dividing the tissue between the fistula and the skin to promote tissue regeneration and hence healing of the fistula. A disadvantage with this procedure is that it causes discomfort and scarring, and usually results in some level of incontinence.

In an alternative procedure, a seton may be used which is passed through the tract of the fistula by use of a fistula probe. The seton is a string often formed out of silicon or rubber that is typically threaded through an eye of the fistula probe. The probe is then passed through the tract of the fistula pulling the seton along so that it extends through the entire length of the fistula. As the probe reaches the wall of the anal canal, the probe is passed through the anus and then removed from the seton such that the two loose ends of the seton can be tied together to form a loop. The seton is typically either left in place long-term and assists in draining any discharge from the fistula or is tied tight to produce a slow form of fistulotomy, that is, division of tissues superficial to the fistula. Further surgical steps to close the fistula may be required after the elimination of the infection.

In an attempt to avoid fistulotomy, an alternative procedure using a fibrin plug has been introduced. The fibrin plug comprises a scaffold of polymeric fibrin that is inserted into the fistula tract to promote tissue in-growth into the scaffold to restore natural tissue healing and formation.

### SUMMARY

The present inventors recognize that there is a need for an implantable device that aids in healing a tunnel wound, e.g., a fistula tract, that reduces the chance of adverse immunological reactions, that reduces the leakage of fluid through the fistula tract, and that reduces the chance of migration or dislodgement of the device. The present inventors further recognize that a disadvantage with existing fibrin plugs is that they tend to fall out of the fistula tract before the end of the 4-to-6-week healing process, even if sutured to bodily tissue.

An implantable device for the treatment of a tunnel wound, e.g., a fistula tract, is provided herein. The device can include a folded length of a suture, string or a seton having a tunnel filler, which may be a fistula filler, comprising decellularized mammalian extracellular matrix (ECM) compressed thereon, which is proximal to a cap member, which may be in the shape of a torus and formed of decellularized mammalian ECM, wherein the suture extends through the cap member, which is proximal to a tamp member, wherein the suture is within an optional hollow of the tamp member, which is proximal to an optional handle member to which the ends of the suture can extend from or attach to, wherein the fold provides a loop that extends from the proximal end of the tunnel filler.

In one embodiment, an implantable device for the treatment of a tunnel would is provided and comprises: a folded length of a suture, string, or seton having a proximal loop and two distal ends, a tunnel filler comprising decellularized mammalian ECM applied to the suture, string or seton, a cap member formed of decellularized mammalian ECM, polycaprolactone, polylactic acid (PLA), polyglycolic acid (PGA), or co-polymers of PLA and PGA, a tamp member, and optionally a handle member, wherein the suture, string or seton transverses through the tunnel filler, the cap member, and the hollow of the tamp member to a connection with the optional handle member, wherein the fold provides a loop that extends from the proximal end of the tunnel filler. In one embodiment, the tunnel filler is radially compressed onto the suture. In one embodiment, the tunnel filler is torsionally compressed onto the suture. In one embodiment, the tunnel filler is an anal fistula filler. In one embodiment, the decellularized mammalian ECM is porcine ECM. In one embodiment, the length of the suture, string or seton is about 40 centimeters (cm) to about 100 cm, e.g., about 50 cm to about 70 cm. In one embodiment, a compressed diameter of the tunnel filler is from about 1 millimeter (mm) to about 6 mm. In one embodiment, the dimensions of the tunnel filler comprise about 4 cm to about 6 cm in length, e.g., about 5 cm in length. In one embodiment, the dimensions of the filler comprise about 8 cm to about 10 cm in length, e.g., about 9 cm in length. In one embodiment, the radial dimensions of the tunnel filler are uniform along its entire length. In one embodiment, the radial dimensions of the tunnel filler gradually increase along its length to create a tapered or conical shaped filler. In one embodiment, the cap member comprises one sheet of compressed ECM. In one embodiment, the cap member comprises two or more sheets of compressed ECM. In one embodiment, the sheets are dried ECM. In one embodiment, the diameter of the cap member, the tamp member, and/or the handle member is from about 1 mm to about 6 mm. In one embodiment, the tunnel filler has a tapered shape.

In one embodiment, an implantable device for the treatment of a tunnel wound is provided and comprises: a wire having a proximal loop and two distal ends, a tunnel filler comprising decellularized mammalian ECM applied to the wire, a cap member formed of decellularized mammalian ECM, polycaprolactone, polylactic acid (PLA), polyglycolic acid (PGA), or co-polymers of PLA and PGA, a tamp member, and optionally a handle member, wherein the wire transverses through the tunnel filler, the cap member, and the tamp member to a connection with the optional handle member, wherein the fold provides a loop that extends from the proximal end of the tunnel filler. In one embodiment, the tunnel filler is radially compressed on the wire. In one embodiment, the tunnel filler is torsionally compressed onto the wire. In one embodiment, the tunnel filler is an anal fistula filler. In one embodiment, the decellularized mammalian ECM is porcine ECM. In one embodiment, the length of the wire is about 40 cm to about 100 cm, e.g., about 50 cm to about 70 cm. In one embodiment, the diameter of the tunnel filler is from about 1 mm to about 6 mm. In one embodiment, the dimensions of the filler comprise about 4 cm to about 6 cm in length, e.g., about 5 cm in length. In one embodiment, the dimensions of the filler comprise about 8 cm to about 10 cm in length, e.g., about 9 cm in length. In one embodiment, the radial dimensions of the tunnel filler are uniform along its entire length. In one embodiment, the radial dimensions of the tunnel filler gradually increase along its length to create a tapered or conical shaped filler. In one embodiment, the cap member comprises one sheet of compressed ECM. In one embodiment, the cap member comprises two or more sheets of compressed ECM. In one embodiment, the sheets are dried ECM. In one embodiment, the diameter of the cap member, the tamp member, and/or the handle member is from about 1 mm to about 6 mm. In one embodiment, the tunnel filler has a tapered shape.

Also provided is a kit that comprises a tunnel filler comprising decellularized mammalian ECM and one or more of: a tunnel brush; a suture, string, seton or wire; a cap member formed of decellularized mammalian ECM; a tamp member, a handle member, or a combination thereof.

In one embodiment, a method to prepare an implantable device for treatment of a tunnel wound is provided and comprises applying or introducing to a suture, string, seton or wire, two or more of: a tunnel filler comprising decellularized mammalian ECM, a cap member formed of decellularized mammalian ECM, a tamp member, or a handle member; or threading a suture, string, seton or wire through a tunnel filler comprising decellularized mammalian ECM, and one or more of: a cap member formed of decellularized mammalian ECM, a tamp member, or a handle member. In one embodiment, the suture, string, seton or wire comprises a loop and two ends.

Further provided is a method of treating a tunnel wound in a mammal, comprising: introducing the device to a proximal or distal end of the tunnel wound in a mammal in need thereof; applying pressure from the tamp member to the cap member to position the tunnel filler in the tunnel wound; removing the tamp member, e.g., over the suture or by severing the suture between the cap member and the tamp member; and affixing the ends of the suture, string, seton or wire at the proximal or distal end of the tunnel wound. In one embodiment, the loop of the suture, string, seton or wire is attached to a tunnel brush. In one embodiment, after urging the tunnel brush through the tunnel wound, thereby debriding the surfaces of the tunnel wound, the loop extends out of a distal end of the fistula. In one embodiment, the method includes affixing the suture, string, seton or wire at the distal end.

Also provided is an implantable device for the treatment of a tunnel wound, which comprises: a length of a suture, string, seton or wire having a distal end and a proximal end, a tunnel filler comprising decellularized mammalian ECM compressed on the suture, string, seton, or wire, which tunnel filler is proximal to a cap member formed of decellularized mammalian ECM and the suture, string, seton or wire is within a center of the cap member, which cap member is proximal to a tamp member, wherein the suture extends within the tamp member, which tamp member is proximal to an optional handle member. In one embodiment, the proximal or the distal end of the suture, string, seton or wire has a loop. In one embodiment, the proximal end and the distal end of the suture, string, seton or wire have a loop.

Also provided is a method of treating a tunnel wound in a mammal, which comprises: introducing the device to a proximal or distal end of the tunnel wound; applying pressure from the tamp member to the cap member to position the tunnel filler in the tunnel wound; severing the suture between the cap member and the tamp member; and affixing the ends of the suture at the proximal end or distal end of the tunnel wound.

These and other examples and features of the present devices and methods will be set forth, at least in part, in the following Detailed Description. This Summary is intended to provide non-limiting examples of the present subject matter—it is not intended to provide an exclusive or exhaustive explanation. The Detailed Description below is included to provide further information about the present devices and methods.

### BRIEF DESCRIPTION OF FIGURES

In the drawings, like numerals can be used to describe similar features and components throughout the several views. The drawings illustrate generally, by way of example, but not by way of limitation, various device and method embodiments discussed in this patent document.

FIG. 1 illustrates a device for the treatment of a tunnel wound, as constructed in accordance with at least one embodiment.

FIGS. 2A-2F illustrate a method of using the device of FIG. 1 to treat a tunnel wound in a mammal, as constructed in accordance with at least one embodiment.

### DETAILED DESCRIPTION

A fistula is an abnormal anastomosis, that is, an abnormal connection between a set of organs or vessels, e.g., where there are two hollow spaces (technically, two epithelialized surfaces), such as blood vessels, intestines, or other hollow organs, or forms from a soft tissue wound cavity which becomes infected and tunnels its way to form a soft body abscess, resulting in the fistula. The connection may take on the form of a tunnel tract which is often difficult to heal or close. Fistulas can be caused by injury or surgery, but they can also result from an infection or inflammation. Fistulas are generally a disease condition, but they may be surgically created for therapeutic reasons.

Surgery is often required to assure adequate drainage of the fistula (so that pus may escape) to minimize abscess formation or the creation of additional tunnelling wounds which could form additional fistulas. Various surgical procedures can be used, most commonly fistulotomy, placement of a seton (a cord that is passed through the path of the fistula to keep it open for draining), or an endorectal flap procedure (where healthy tissue can be pulled over the internal side of the fistula to keep feces or other material from reinfecting the channel). Current treatment often involves filling the fistula with fibrin glue or plugging it with plugs made of porcine small intestine submucosa. Surgery for fistulae, e.g., anorectal fistulae, is not without side effects, including recurrence, reinfection, and incontinence.

The present disclosure provides a device including a tunnel filler formed of decellularized mammalian ECM, and one or more of an ECM cap member, a tamp member, a handle member, or any combination thereof, suspended on or affixed to a structure such as a suture, string or seton.

The native ECM is a complex network of structural and functional proteins that form tissue-specific architectures. The native ECM includes secreted products of resident cells in each tissue and organ. The matrix molecules include structural and functional proteins, glycoproteins, and glycosaminoglycans. The resident cells of the native ECM, besides producing ECM, receive signals therefrom, allowing for tissue development and/or homeostasis. Those properties are the basis for the use of ECM-based materials in tissue engineering and regenerative medicine. Because ECM provides a naturally occurring and highly conserved substrate for cell viability and growth that has reduced immunogenicity, ECM-based substrates having individual ECM components or of whole decellularized tissues have been used in a wide range of applications in both preclinical and clinical settings.

Optionally, the ECM can be cross-linked to stabilize its biological matrix and slow its degradation post-implantation. In one example, formaldehyde is used to cross-link the ECM. Cross-linking with formaldehyde can increase the durability of the biological matrix, making it resistant to enzymatic degradation.

The disclosure provides for a tunnel filler, e.g., for an anal fistula, comprising decellularized mammalian ECM, e.g., derived from a mammalian organ or tissue, and components for delivering the tunnel filler into the fistula.

The mammalian organ may be from a pig or human. The mammalian organ may be a liver, muscle, lung, spleen, kidney, or heart. The shape of the tunnel filler may be, for example, a cube, rectangular prism, or an irregular strip. The shape of the tunnel filler may be configured for an anal, vaginal, biliary, gastrointestinal, bladder or esophageal fistula.

The tunnel filler formed from ECM may be sterilized by a dose of radiation from about 5 kilo Grays (kGy) to about 50 kGy. The radiation may be E-beam radiation or gamma radiation. The radiation may be thermal or UV radiation.

The tamp member or handle member may be formed of plastic, metal, a composite, a biologic material, or any combination thereof. The tamp member may be a cylindrical tube, a triangular tube, a square tube, or a rectangular tube.

The device may be employed to treat a disease or condition in a mammal such as human by: introducing the device into a tunnel wound of a mammal and after tunnel filler placement, removing the tamp member and/or handle member, and optionally the cap member, and securing the tunnel filler in the tunnel wound by securing the suture, string or seton at the proximal and distal ends of the tunnel wound.

### Exemplary Sources of Organs and Tissues for Decellularized ECM

A tissue is a group of cells with a common structure and function, e.g., epithelial tissue, connective tissue, muscle tissue (skeletal, cardiac, or smooth muscle), and nervous tissue, and includes a pliable sheet that covers or lines or connects organs. An organ is a collection of tissues (two or more) joined in structural unit to serve a common function. Organs include but are not limited to the brain, liver, pancreas, bone, spleen, heart, stomach, kidney, lungs, whole muscles, thymus, anus, and intestine. As used herein, an organ includes perfusable whole organs, or parts of an organ, or vascularized structures thereof, and a tissue includes any structures that contain vascularized tissues, e.g., a trachea.

In one embodiment, a portion of a decellularized organ or tissue or ECM thereof, is employed in the tunnel filler, e.g., a decellularized porcine liver ECM or a portion thereof. The ECM of an organ or tissue, or a vascularized portion thereof, may be obtained from any source including, without limitation, heart, liver, lungs, skeletal muscles, brain, pancreas, spleen, kidneys, uterus, eye, spinal cord, intestine, omentum, whole muscle, or bladder, or any portion thereof (e.g., an aortic valve, a mitral valve, a pulmonary valve, a tricuspid valve, a pulmonary vein, a pulmonary artery, coronary vasculature, septum, a right atrium, a left atrium, a right ventricle, or a left ventricle). A solid organ refers to an organ that has a “substantially closed” vasculature system. A “substantially closed” vasculature system with respect to an organ means that, upon perfusion with a liquid, most of the liquid is contained within the solid organ or runs through the native vascular structures and does not leak out of the solid organ, assuming the major vessels are cannulated, ligated, or otherwise restricted. Despite having a “substantially closed” vasculature system, many of the organs listed above have defined “entrance” and “exit” vessels which are useful for introducing and moving the liquid throughout the organ during perfusion. In addition, other types of vascularized organs or tissues such as, for example, all or portions of joints (e.g., knees, shoulders, or hips), anus, trachea, or spinal cord, can be perfusion decellularized. Further, avascular tissues such as, for example, cartilage or cornea, may be decellularized when part of larger vascularized structures.

Perfusion decellularized ECM of organs with a substantially closed vascular system are useful because perfusion decellularization preserves the intact matrix and microenvironment, including an intact vascular and microvascular system, that vascular system, or ducts or other conduits, may be utilized to deliver nutrients and/or differentiation or maintenance factors. Nutrients and/or other growth factors may be delivered by other means, e.g., injection, or passive means, or a combination thereof.

### Decellularization of Organs or Tissues

Decellularization generally includes the following steps: stabilization of the solid organ, e.g., a vascularized structure thereof, or tissue, decellularization of the solid organ or tissue, washing the decellularized solid organ or tissue, degradation of any DNA remaining in/on the decellularized organ or tissue, and optionally disinfection of the decellularized organ or tissue and/or homeostasis of the decellularized organ or tissue.

The initial step in decellularizing an organ's vascularized structure or tissue is to cannulate the organ or tissue. The vessels, ducts, and/or cavities of an organ or tissue may be cannulated using methods and materials known in the art. Next, the cannulated organ vascularized structure or tissue is perfused with a cellular disruption medium. Perfusion through an organ can be multi-directional (e.g., antegrade and retrograde).

Langendorff perfusion of a heart is routine in the art, as is physiological perfusion (also known as four chamber working mode perfusion). See, for example, Dehnert, The Isolated Perfused Warm-Blooded Heart According to Langendorff, In Methods in Experimental Physiology and Pharmacology: Biological Measurement Techniques V. Biomesstechnik-Verlag March GmbH, West Germany, 1988.

Briefly, for Langendorff perfusion, the aorta is cannulated and attached to a reservoir containing physiological solution to allow the heart to function outside of the body for a specified duration of time. To achieve perfusion decellularization, the protocol has been modified to perfuse a cellular disruption medium delivered in a retrograde direction down the aorta either at a constant flow rate delivered, for example, by an infusion or roller pump or by a constant hydrostatic pressure pump. In both instances, the aortic valves are forced shut and the perfusion fluid is directed into the coronary ostia (thereby perfusing, via antegrade, the entire ventricular mass of the heart), which then drains into the right atrium via the coronary sinus. For working mode perfusion, a second cannula is connected to the left atrium and perfusion can be changed to retrograde.

In one embodiment, a physiological solution includes phosphate buffer saline (PBS). In one embodiment, the physiological solution is a physiologically compatible buffer supplemented with, e.g., nutritional supplements (for instance, glucose).

Methods are known in the art for perfusing other organs or tissues. By way of example, the following references describe the perfusion of lung, liver, kidney, brain, and limbs. Van Putte et al., Ann. Thorac. Surg., 74 (3): 893 (2002); den Butter et al., Transpl. Int., 8:466 (1995); Firth et al., Clin. Sci. (Lond.), 77 (6): 657 (1989); Mazzetti et al., Brain Res., 999 (1): 81 (2004); Wagner et al., J. Artif. Organs, 6 (3): 183 (2003).

One or more cellular disruption media may be used to decellularize an organ or tissue. A cellular disruption medium generally includes at least one detergent such as but not limited to SDS, PEG, CHAPS or Triton X. A cellular disruption medium can include water such that the medium is osmotically incompatible with the cells. Alternatively, a cellular disruption medium can include a buffer (e.g., PBS) for osmotic compatibility with the cells. Cellular disruption media also may include enzymes such as, without limitation, one or more collagenases, one or more dispases, one or more DNases, or a protease such as trypsin. In some instances, cellular disruption media also or alternatively may include inhibitors of one or more enzymes (e.g., protease inhibitors, nuclease inhibitors, and/or collagenase inhibitors).

In certain embodiments, a cannulated organ or tissue may be perfused sequentially with two different cellular disruption media. For example, the first cellular disruption medium may include an anionic detergent such as SDS and the second cellular disruption medium can include an ionic detergent such as Triton X. Following perfusion with at least one cellular disruption medium, a cannulated organ or tissue may be perfused, for example, with wash solutions and/or solutions containing one or more enzymes such as those disclosed herein.

Alternating the direction of perfusion (e.g., antegrade and retrograde) may assist in decellularizing the entire organ or tissue. Decellularization generally decellularizes the organ from the inside out, resulting in very little damage to the ECM. An organ or tissue may be decellularized at a suitable temperature between 4° C. and 40° C. Depending upon the size and weight of an organ or tissue and the particular detergent(s) and concentration of detergent(s) in the cellular disruption medium, an organ or tissue generally is perfused from about 0.05 hours to about 5 hours, per gram of solid organ or tissue (generally >50 grams), or about 2 hours to about 12 hours, per gram of solid organ or tissue for organs (generally <50 grams), with cellular disruption medium. Including washes, an organ may be perfused for up to about 0.75 hours to about 10 hours per gram of solid organ or tissue (generally >50 grams), or about 12 hours to about 72 hours, per gram of tissue (generally <50 grams). Decellularization time is dependent upon the vascular and cellular density of the organ or tissue with limited scaling for overall mass. Therefore, as general guidance the time ranges and masses above are provided. Perfusion generally is adjusted to physiologic conditions including pulsatile flow, rate, and pressure.

A decellularized organ or tissue has the ECM component of all or most regions of the organ or tissue, including ECM components of the vascular tree. ECM components can include any or all of the following: fibronectin, fibrillin, laminin, elastin, members of the collagen family (e.g., collagen I, III, and IV), ECM associated growth proteins including growth factors and cytokines, glycosaminoglycans, ground substance, reticular fibers and thrombospondin, which can remain organized as defined structures such as the basal lamina.

The morphology and the architecture of the ECM is maintained during and following the process of decellularization. “Morphology” as used herein refers to the overall shape of the organ, tissue or of the ECM, while “architecture” as used herein refers to the exterior surface, the interior surface, and the ECM therebetween.

The morphology and architecture of the ECM may be examined visually and/or histologically. For example, the basal lamina on the exterior surface of a solid organ or within the vasculature of an organ or tissue should not be removed or significantly damaged due to perfusion decellularization. In addition, the fibrils of the ECM should be similar to or significantly unchanged from that of an organ or tissue that has not been decellularized.

### Controlled System for Decellularizing an Organ or Tissue

A system for decellularizing an organ or tissue generally includes at least one cannulation device for cannulating an organ or tissue, and a perfusion apparatus for perfusing the organ or tissue through the cannula(s). Cannulation and perfusion are well-known techniques in the art. A cannulation device generally includes size-appropriate hollow tubing for introducing into a vessel, duct, and/or cavity of an organ or tissue. Typically, one or more vessels, ducts, and/or cavities are cannulated in an organ. A perfusion apparatus can include a holding container for the liquid (e.g., a cellular disruption medium) and a mechanism for moving the liquid through the organ (e.g., a pump, air pressure, gravity) via the one or more cannulae. The sterility of an organ or tissue during decellularization can be maintained using a variety of techniques known in the art such as controlling and filtering the air flow and/or perfusing with, for example, antibiotics, anti-fungals or other anti-microbials to prevent the growth of unwanted microorganisms.

A system to decellularize organ or tissues as described herein can possess the ability to monitor certain perfusion characteristics (e.g., pressure, volume, flow pattern, temperature, gases, pH), mechanical forces (e.g., ventricular wall motion and stress), and electrical stimulation (e.g., pacing). As the vascular bed changes over the course of decellularization (e.g., vascular resistance, volume), a pressure-regulated perfusion apparatus is advantageous to avoid large fluctuations. The effectiveness of perfusion can be evaluated in the effluent and in tissue sections. Perfusion volume, flow pattern, temperature, partial O₂ and CO₂ pressures and pH can be monitored using standard methods.

Sensors can be used to monitor the system and/or the organ or tissue. In addition to having sensors for monitoring such features, a system for decellularizing an organ or tissue also can include means for maintaining or adjusting such features. To help ensure stable conditions (e.g., temperature), the chambers, reservoirs and tubing can be water jacketed.

A system may be controlled by a computer-readable storage medium in combination with a programmable processor (e.g., a computer-readable storage medium as used herein has instructions stored thereon for causing a programmable processor to perform particular steps). For example, such a storage medium, in combination with a programmable processor, may receive and process information from one or more of the sensors. Such a storage medium in conjunction with a programmable processor also can transmit information and instructions back to the bioreactor and/or the organ or tissue.

In one embodiment, the weight of an organ or tissue may be entered into a computer-readable storage medium as described herein, which, in combination with a programmable processor, can calculate exposure times and perfusion pressures for that particular organ or tissue. Such a storage medium may record preload and afterload (the pressure before and after perfusion, respectively) and the rate of flow. In this embodiment, for example, a computer-readable storage medium in combination with a programmable processor can adjust the perfusion pressure, the direction of perfusion, and/or the type of perfusion solution via one or more pumps and/or valve controls.

### Exemplary Devices and Methods

For example, the tunnel filler formed of decellularized tissue, e.g., after irradiation, may be introduced to a delivery device such as one for surgical placement of a tunnel filler, e.g., a product that takes on the shape of a fistula or other tunnel wound and/or that displaces the area of the fistula in order to fit inside the fistula. The device may be inserted into the fistula tract and the tunnel filler may be deployed by pulling, pushing or otherwise expelling the tunnel filler into the tract. The tamp member and/or handle member of the device can then be removed from the tract, e.g., over the suture or by cutting the suture between the cap member and the handle member, or between the tunnel filler and the cap member, leaving behind the tunnel filler and optionally the cap member within the fistula tract.

A kit having the tunnel filler and suture and optionally the cap member, tamp member, and/or handle member can be in packaging, e.g., a pouch, tray, vial, or sterile container, which kit or individual components thereof may be subjected to a selected dose of radiation. The tunnel filler in the kit may be formed of decellularized tissue from a pig or human organ. In one embodiment, the organ can be a liver, muscle, lung, spleen, kidney, or heart. In one embodiment, the dose of radiation can be about 5 kGy to about 50 kGy. In one embodiment, the dose of radiation can be about 5 kGy to about 15 kGy. In one embodiment, the dose of radiation can be about 15 kGy to about 25 kGy. In one embodiment, the dose of radiation can be about 25 kGy to about 50 kGy. In one embodiment, the radiation can be E-beam radiation or gamma radiation. In one embodiment, the radiation can be thermal or UV radiation. In one embodiment, before irradiation, the decellularized ECM can be dried. In one embodiment, after irradiation, the decellularized extracellular matrix can be dried. In one embodiment, the dry tunnel filler can be about 0.25 cm×0.25 cm, e.g., about 1 cm×1 cm to about 60 cm×60 cm (length by width). In one embodiment, the dry tunnel filler can be about 0.5 cm×0.5 cm×0.5 cm to about 30 cm×30 cm×30 cm. In one embodiment, the dry tunnel filler thereof can be about 1 cm×1 cm (length times width) to about 10 cm×10 cm. In one embodiment, the dry tunnel filler can be about 1 cm×2 cm to about 10 cm×12 cm. In one embodiment, the dry tunnel filler can be about 1 cm×6 cm to about 3 cm×20 cm. In one embodiment, the dry tunnel filler for anal uses can be about 0.5 cm×4 cm to about 3 cm×10 cm, for vaginal uses can be about 0.5 cm×4 cm to about 3 cm×10 cm, for biliary uses can be about 0.2 cm×2 cm to about 3 cm×1 cm, for gastroenterological use can be about 0.2 cm×2 cm to about 3 cm×10 cm, for bladder uses can be about 0.5 cm×4 cm to about 3 cm×10 cm, for esophageal uses can be about 0.2 cm×2 cm to 3 cm×10 cm. In one embodiment, the tunnel filler can be obtained from a decellularized mammalian organ or tissue. In one embodiment, the tunnel filler can be obtained from the mammalian organ or tissue before decellularization. In one embodiment, the decellularized ECM from the mammalian organ or tissue or the portion thereof prior to radiation, can be inflated with a gas or vapor. In one embodiment, the portion, prior to and after radiation, has the shape of a cube, rectangular prism, or an irregular strip. In one embodiment, the cube can be about 1 cm×1 cm to about 10 cm×10 cm. In one embodiment, the rectangular prism can be about 1 cm×2 cm to about 5 cm to about 12 cm. In one embodiment, an irregular strip about 1 cm×6 cm to about 3 cm to about 20 cm. In one embodiment, the tunnel filler can be about 1 cm×1 cm to about 60 cm×60 cm. In one embodiment, the tunnel filler can be about 0.5 cm×0.5 cm×0.5 cm to about 30 cm×30 cm×30 cm. In one embodiment, the tunnel filler can be about 1 cm×1 cm to about 10 cm×10 cm. In one embodiment, the tunnel filler can be about 1 cm×2 cm to about 10 cm×12 cm. In one embodiment, the tunnel filler can be about 1 cm×6 cm to about 3 cm×20 cm. In one embodiment, the tunnel filler can be subjected to dehydration.

In one embodiment, the decellularized material is compressed into a cylindrical rod. For example, a rectangular, three-dimensional piece that starts at about 2 cm×2 cm×9 cm long is radially compressed down to an about 3 mm diameter cylinder (9 cm long) or radially compressed down to an about 6 mm diameter cylinder (9 cm long).

Decellularized organs or tissues, or portions thereof, such as liver, lung, muscle, spleen, kidney, or heart can thus be cut into various shapes. In some embodiments, a dose of radiation can be applied to the shape or to the decellularized organ or tissue before it is cut into a shape(s), from about 1 kGy to about 100 kGy, from about 5 kGy to about 100 kGy, from about 10 kGy to about 100 kGy, from about 15 kGy to about 100 kGy, from about 20 kGy to about 100 kGy, from about 25 kGy to about 100 kGy, from about 30 kGy to about 100 kGy, from about 35 kGy to about 100 kGy, from about 40 kGy to about 100 kGy, from about 45 kGy to about 100 kGy, from about 50 kGy to about 100 kGy, from about 55 kGy to about 100 kGy, from about 60 kGy to about 100 kGy, from about 65 kGy to about 100 kGy, from about 70 kGy to about 100 kGy, from about 75 kGy to about 100 kGy, from about 80 kGy to about 100 kGy, from about 85 kGy to about 100 kGy, from about 90 kGy to about 100 kGy, or from about 95 kGy to about 100 kGy.

In one embodiment, the decellularized organ or tissue matrix or portion of an organ or tissue matrix, is cut prior to irradiation to provide for desired three-dimensional portions of the matrix. In one embodiment, the decellularized organ or tissue matrix or portion of an organ or tissue matrix is irradiated then cut into desired three-dimensional portions of the matrix. In one embodiment, the decellularized organ or tissue matrix or portion of an organ or tissue matrix is cut into desired three-dimensional portions of the matrix then irradiated. In one embodiment, the irradiated decellularized tissue or portion of an organ can be used in treatment of fistulas. In one embodiment, the irradiated decellularized tissue or portion of an organ can be used as a tunnel filler, e.g., a fistula filler. In one embodiment, the decellularized tissue or portion of an organ can be inflated with a gas, including ambient air, or vapor before irradiation or drying.

Also disclosed herein are methods of preparing a tunnel filler delivery device. The method can include providing a tunnel filler comprising ECM; providing a suture; providing a cap member comprising ECM; providing a tamp member; and optionally providing a handle member; and assembling the tunnel filler, the cap member, the tamp member, and optional handle member on the suture. In one embodiment, the tamp member and handle member are a single component. In one embodiment, the tamp member or handle member can be formed of plastic, metal, a composite, a biologic material, or combination thereof. In one embodiment, the tamp member can be a cylindrical tube, a triangular tube, a square tube, or a rectangular tube. In one embodiment, the device can further comprise a handle member. In one embodiment, two ends of the suture are threaded through the tunnel filler, then the cap member, then the tamp member, leaving a loop extending from the end of the tunnel filler. In one embodiment, one end of a suture is threaded through the tamp member, then the cap member, then the tunnel filler, and then threaded back through the tunnel filler, the cap member, and the tamp member, leaving a loop extending from the end of the filler. In one embodiment, one end of a suture is threaded through the tunnel filler, then the cap member, then the tamp member, and then threaded back through the tunnel filler, the cap member, and the tamp member, leaving a loop extending from the tamp member. The proximal end of the tunnel filler with two loose ends of the suture may be tied off so that a tunnel brush having a suture or fistula seton affixed thereto can be attached to the tied end of the suture. The distal end of the tamp member, or handle member if present, with two loose ends of the suture may be tied off.

Uses for the device can include a method of treating a mammal in need of a tunnel filler, e.g., where the mammal may be in need of anal, vaginal, biliary, gastrointestinal, bladder or esophageal fistula repair, or abnormal connections caused by infection tunnelling and forming an abscess or cavity or caused by trauma wounds.

Also disclosed herein are kits that can comprise a tunnel filler and a tunnel brush. In some embodiments, a kit can comprise a delivery device comprising a tunnel filler and a suture. In some embodiments, a kit can comprise a tunnel filler and one or more of a suture, a cap member, a tamp member, a handle member, a tunnel brush, or a combination thereof, in a sealed container.

Disclosed herein are systems which can be composed of a device containing a tunnel filler that may be inserted into a tunnel wound, e.g., a fistula tract. The delivery device allows for placement of a tunnel filler into tunnel wound. The device also allows for easier placement of the tunnel filler into the tunnel wound. Placement of the tunnel filler can be aided by the device which allows the tunnel filler to be placed within the tunnel wound prior to removal of a cap member, a tamp member, and/or a handle member. The tamp member or handle member may be formed of a polymer including but not limited to polytetrafluoroethylene, polypropylene, polyethylene, polystyrene, nylon, polyetheretherketone, or polyurethane. In one embodiment, the tamp member or handle member may be composed of plastic, metal, composite, biologic material, or combination thereof. In one embodiment, the tamp member may be a cylindrical tube, triangular tube, square tube, or rectangular tube. In one embodiment, the tamp member or handle member may have non-rigid walls. In one embodiment, the device may be used to deploy the tunnel filler into anal fistulas, vaginal fistulas, biliary fistulas, gastrointestinal fistulas, bladder fistulas, esophageal fistulas, and soft tissue wound fistulas.

### Exemplary Uses for the Tunnel Filler

The decellularized tissue forms a product useful to treat, for example, diseases of the eye, adnexa, ear, and mastoid process including but not limited to lacrimal fistula, mastoid fistula craniofistula, e.g., between the intracranial space and a paranasal sinus, labyrinthine fistula, perilymph fistula, or preauricular fistula; diseases of the circulatory system including but not limited to coronary arteriovenous fistula, arteriovenous fistula, e.g., of the pulmonary vessels cerebral arteriovenous fistula, acquired, or fistula of an artery; diseases of the respiratory system including but not limited to pyothorax with fistula or tracheoesophageal fistula; diseases of the digestive system including but not limited to duodeno biliary fistula, e.g., salivary gland fistula, fistula of stomach and duodenum, gastrocolic fistula, gastrojejunocolic fistula, enterocutaneous fistula, gastric fistula from the stomach to the skin surface, fistula of appendix, anal and rectal fissures and fistulas, e.g., anal fistula or anorectal fistula (fecal fistula, fistula-in-ano), fistula of intestine, e.g., enteroenteral fistula: between two parts of the intestine, fistula of gallbladder or fistula of bile duct, e.g., biliary fistula, or pancreatic fistula: between the pancreas and the exterior via the abdominal wall; diseases of the musculoskeletal system and connective tissue including fistula of joint; diseases of the urogenital system including but not limited to vesicointestinal fistula, urethral fistula, fistula of nipple, fistulae involving female genital tract/obstetric fistula including, e.g., vesicovaginal fistula, cervical fistula, enterovaginal fistula: between the intestine and the vagina, rectovaginal fistula, other female intestinal-genital tract fistulae, or female genital tract-skin fistulae; congenital malformations, deformations and chromosomal abnormalities including but not limited to sinus, fistula and cyst of branchial cleft, congenital preauricular fistula, portal vein-hepatic artery fistula, congenital fistula of lip, congenital fistula of salivary gland, congenital absence, atresia and stenosis of rectum with fistula, congenital absence, atresia and stenosis of anus with fistula, congenital fistula of rectum and anus, congenital fistulae between uterus and digestive and urinary tracts, or congenital rectovaginal fistula; or external causes including but not limited to traumatic arteriovenous fistula or persistent postoperative fistula.

For example, a delivery device can be inserted in an internal opening and then pulled through a tunnel wound, e.g., following insertion of a tunnel brush or fistula seton that is attached to the device, until light resistance can be met and then sutured securely in the primary opening. Excess decellularized tissue from the external opening may be trimmed at the skin level. Prior to delivery, the tunnel wound may be irrigated with hydrogen peroxide or other anti-microbial or antibiotic solution before insertion. The external opening may be partially open as this is the path that allows drainage and prevents a closed-space infection. The maturing of the tunnel wound, making the wall more fibrotic, which results in increased healing, may minimize sepsis and facilitate wound closure when used in conjunction with other procedures.

### Exemplary Embodiments

An implantable device for the treatment of a fistula is provided. In one embodiment, the device includes a folded length of a suture having a tunnel filler comprising decellularized mammalian ECM compressed thereon, which is proximal to a cap member formed of decellularized mammalian ECM wherein the suture passes through the center of the cap member, which is proximal to a tamp member, wherein the suture passes through the lumen of the tamp member, which is proximal to an optional handle member, wherein the ends of the suture extend from the distal end, wherein the fold provides a loop that extends from the proximal end of the filler. In one embodiment, the device includes a folded length of a suture having a proximal loop and two distal ends, a tunnel filler comprising decellularized mammalian ECM applied to the suture, a cap member formed of decellularized mammalian ECM, a tamp member, and optionally a handle member, wherein the suture transverses the tunnel filler, the cap member, the tamp member and the optional handle member, wherein the ends of the suture extend from the distal end of the tamp member or the optional handle member, wherein the fold provides a loop that extends from the proximal end of the filler. In one embodiment, the tunnel filler is radially compressed on the suture. In one embodiment, the tunnel filler is torsionally compressed on the suture. In one embodiment, the tunnel filler is an anal fistula filler. In one embodiment, the ECM is porcine ECM. In one embodiment, the length of the suture is about 5 to about 10 inches (about 12.7 cm to about 25.4 cm) or about 12 to about 24 inches (e.g., about 30 cm to about 60 cm). In one embodiment, the diameter of the tunnel filler is from about 1 mm to about 6 mm. In one embodiment, the length of the tunnel filler is from about 2 cm to about 12 cm. In one embodiment, the cap member comprises one sheet of compressed ECM. In one embodiment, the cap member comprises two or more sheets of compressed ECM. In one embodiment, the sheets are dried ECM. In one embodiment, the sheets are porcine ECM. In one embodiment, the diameter of the cap member, the tamp member or handle member is from about 1 mm to about 6 mm.

Further provided is a kit comprising a tunnel filler comprising decellularized mammalian ECM and one or more of: a tunnel brush; a suture; a cap member formed of decellularized mammalian ECM; or a tamp member or a handle member, or any combination thereof.

Also provided is a method to prepare an implantable device for treatment of a fistula, comprising: introducing to a suture a tunnel filler comprising decellularized mammalian ECM, and one or more of a cap member formed of decellularized mammalian ECM, a tamp member or a handle member. In one embodiment, the suture comprises a loop and two ends.

A method of treating a fistula in a mammal is provided, comprising: introducing the device to a proximal end of a fistula in a mammal in need thereof; applying pressure from the tamp member to the cap member to position the filler in the fistula; removing the tamp member over the suture; and affixing the ends of the suture at the proximal end of the fistula. In one embodiment, the loop of the suture is attached to a tunnel brush. In one embodiment, after introducing the device the loop extends out of a distal end of the fistula. In one embodiment, the method includes affixing the suture at the distal end. In one embodiment, the device is introduced by introducing the tunnel brush to the fistula. In one embodiment, the introduction of the tunnel brush debrides the exterior surface of the fistula.

In one embodiment, an implantable device for the treatment of a fistula is provided comprising: a length of a suture having a distal end and a proximal end, a tunnel filler comprising decellularized mammalian ECM compressed on the suture, which tunnel filler is proximal to a cap member formed of decellularized mammalian ECM and the suture is within a hollow of the cap member, which cap member is proximal to a tamp member, wherein the suture is within a hollow of the tamp member, which tamp member is proximal to an optional handle member. In one embodiment, the proximal or the distal end of the suture has a loop. In one embodiment, the proximal end and the distal end of the suture have a loop.

In one embodiment, a method of treating a fistula in a mammal includes introducing the device to a proximal end of a fistula in a mammal in need thereof; applying pressure from the tamp member to the cap member to position the filler in the fistula; removing the tamp member over the suture; and affixing the ends of the suture at the proximal end or a distal end of the fistula.

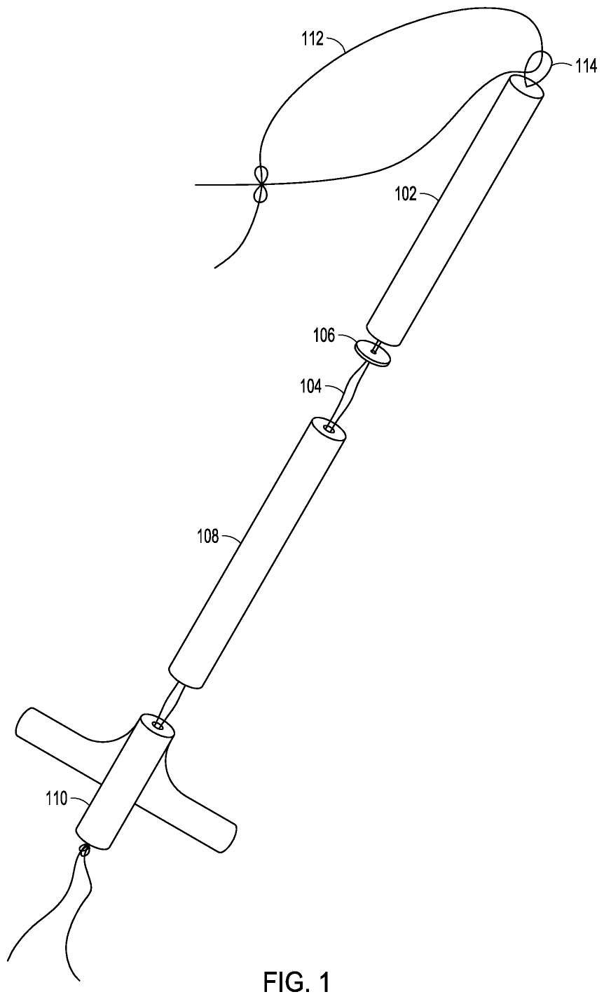

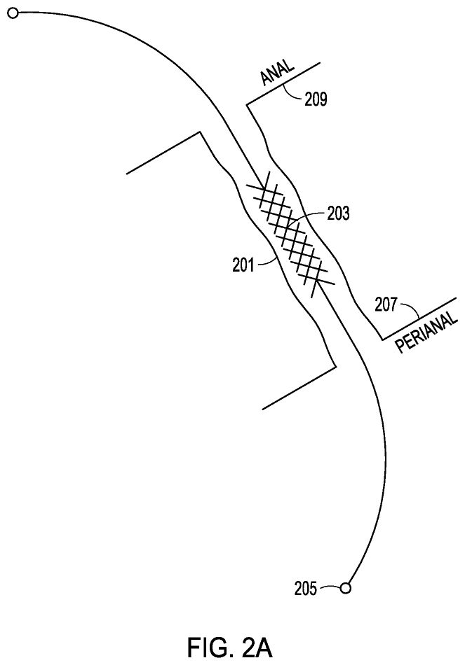

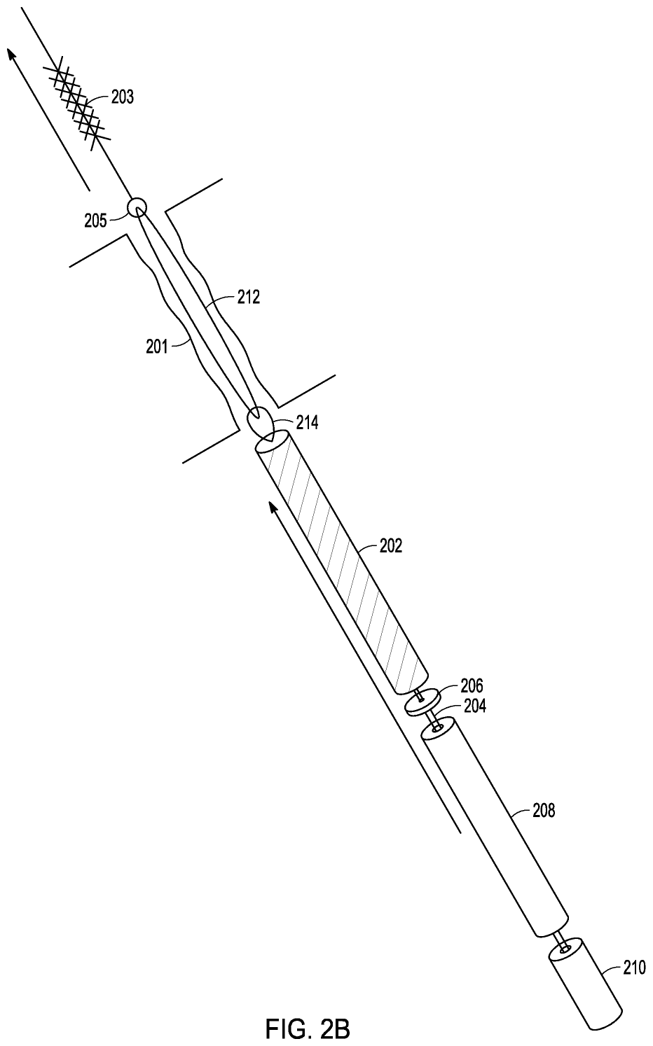

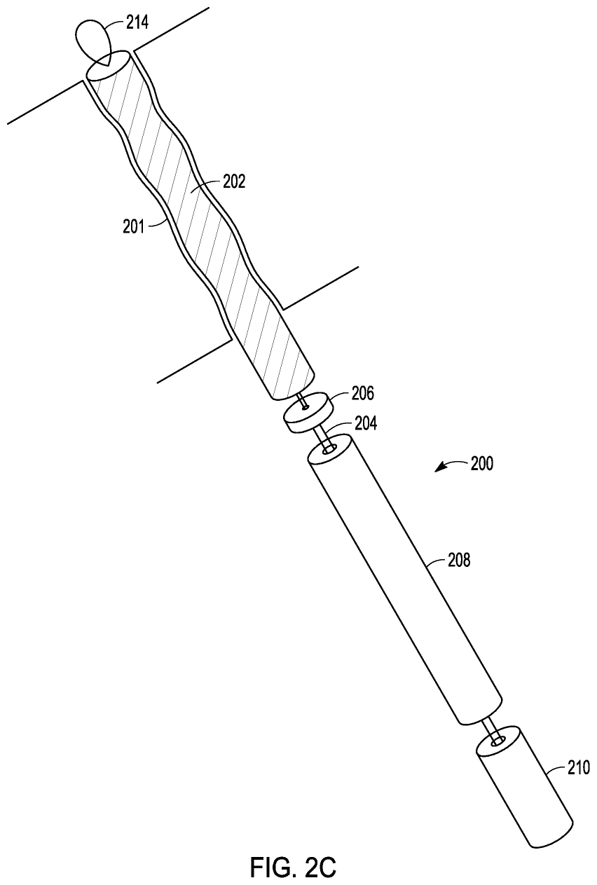

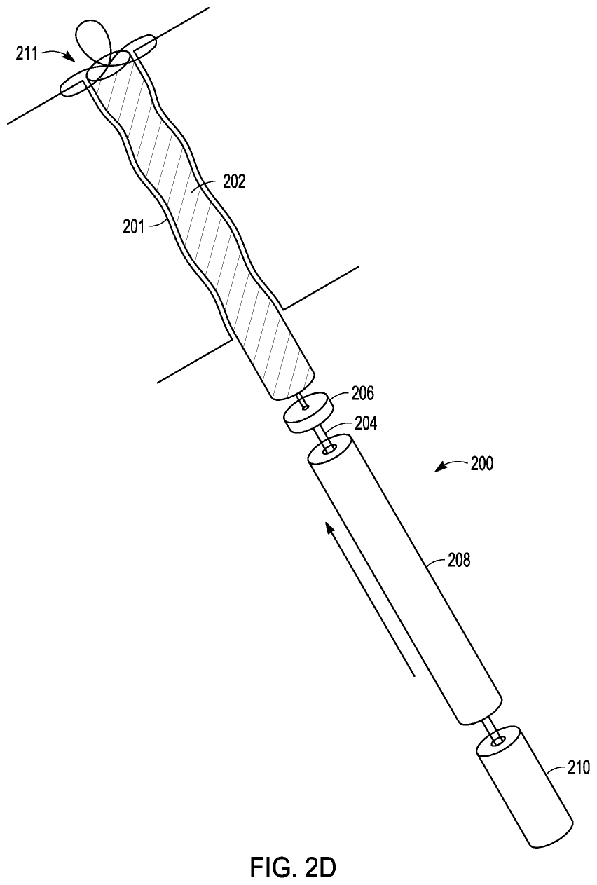

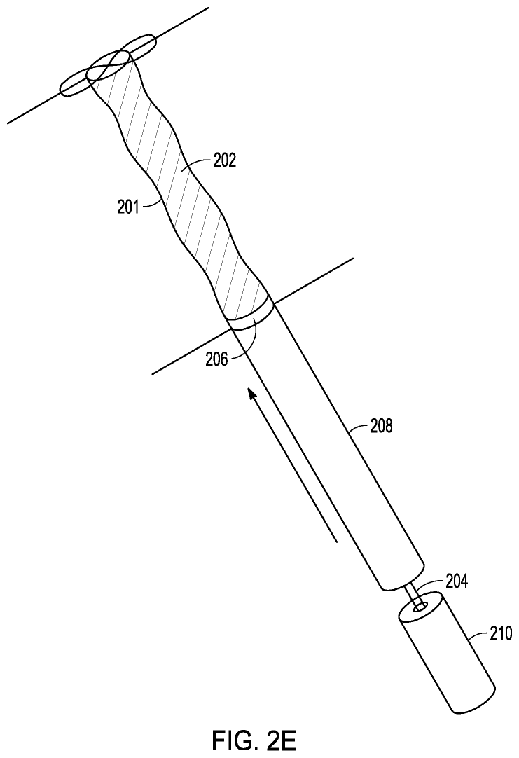

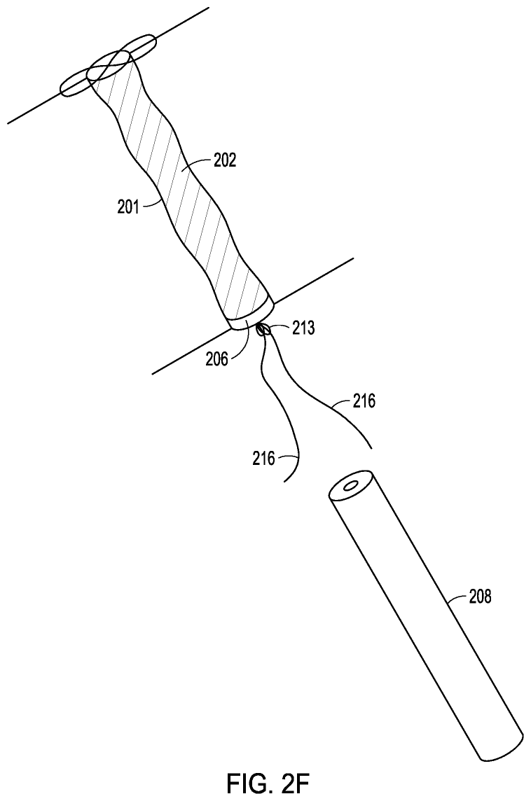

The invention will be described by the following non-limiting example, which is illustrated in FIG. 1 and FIGS. 2A-2F.

A tunnel filler 102, 202, e.g., a fistula filler, is provided that is derived, in one embodiment, from a decellularized mammalian organ or portion thereof, such as a decellularized porcine liver or portion thereof, for the management and treatment of a tunnel wound, e.g., an anal fistula. In one embodiment, a kit is provided that includes a delivery device 100, 200 for the tunnel filler 102, e.g., preloaded with the tunnel filler and/or the tunnel filler optionally with components of the delivery device, and optionally a tunnel brush 203, e.g., a fistula brush, for the preparation of the fistula site and/or to aid in delivery of the device.

In one embodiment the tunnel filler 102, 202 includes a portion of dry decellularized porcine liver, e.g., in the shape of a block or cylinder, threaded and compressed onto a folded length of resorbable suture 104, 204. Distal to the tunnel filler 102, 202 is a thin flat cap member 106, 206 of dried decellularized porcine liver that holds the tunnel filler 102, 202 in place once the device has been delivered within the tunnel wound tract 201. Distal to the cap member 106, 206, are two members—a tamp member 108, 208 and a handle member 110, 210, both optionally formed of plastic—that are positioned over or connected to the suture 104, 204.

In one embodiment, the proximal end of the device 100, 200 (which will traverse the tunnel wound tract 201 from the proximal to the distal end of the wound) includes a secondary loop of suture 112, 212 that allows for delivery of the device to the tunnel wound. After preparation of the tunnel wound tract 201 using the tunnel brush 203, the secondary loop of suture 112, 212 will be attached to the perianal end 205 of the tunnel brush 203 so that as the tunnel brush is removed, the tunnel filler containing device 100, 200 is pulled into the tunnel wound tract 201 through a perianal opening 207, for example. Once the device 100, 200 reaches the anal opening 209 of the tunnel wound tract 201, for example, the secondary loop of suture 112, 212 is removed and the anal side opening 209 of the tunnel wound tract 201 is sutured closed over the end of the device with the closing suture 211 flowing through the loop 114, 214 of the device's integrated core suture 104, 204. Once the device 100, 200 has been secured on the anal end 209 of the tunnel wound tract 201, the tamp member 108, 208 aids in delivering the tunnel filler 102, 202 to the tunnel wound site (e.g., the handle member 110, 210 of the device provides leverage during this process). Once the tunnel filler 102, 202 is delivered, in one embodiment, the suture 104, 204 is cut between the tamp member 108, 208 and the handle member 110, 210 and both components are removed from the device 100, 200. In one embodiment, the suture 104, 204 is cut between the cap member 106, 206 and the tamp member 108, 208, and the tamp member 108, 208 and the handle member 110, 210 are removed from the device 100, 200. At this point, any excess tunnel filler 102, 202 material can be cut away, in one embodiment, and the decellularized cap member 106, 206 is left in place. Once all excess tunnel filler 102, 202 material has been removed, the cap member 106, 206 is positioned against the perianal end 207 of the remaining material, and a knot 213 tied in the two free suture ends 216 holds the decellularized material in place. The tunnel filler 102, 202 may be hydrated in situ by flushing the opening with a physiologically compatible liquid such as sterile saline or lactated Ringer's solution. Normal body fluids may also assist in the hydration of the device. This wetting process allows the tunnel filler 102, 202 to slightly swell and maximize contact with the neighboring tissue within the tunnel wound tract 201.

### Exemplary Tunnel Filler Preparation

In one embodiment, the tunnel filler is prepared from porcine liver that has been decellularized, suspended, inflated, and dried (see, e.g., U.S. Pat. No. 9,974,814, the disclosure of which is incorporated by reference herein in its entirety). The resulting decellularized material, which is dry, porous, and spongy, is cut into segments which can subsequently be loaded onto a length of suture. These segments can, in one embodiment, be about 5 cm to about 10 cm in length and about 1 cm to about 2 cm in width and height (e.g., prior to compression). Once on the suture, the decellularized material may be compressed down to a diameter of about 3 mm to about 6 mm. In one embodiment, the decellularized material is about 5 cm in length with an about 3 mm diameter (dry) or an about 9 cm in length with an about 3 mm diameter (dry). Once deployed and wetted in the tunnel wound tract, the material can swell to approximately two-fold.

In one embodiment, the length of suture is folded over, and the double length is threaded through the tunnel filler portion of the device.

The decellularized cap member, in one embodiment, may be dried, compressed decellularized liver pieces cut into disks of the approximate diameter of the tunnel filler. The cap member may be formed from decellularized porcine liver compressed into two dimensional sheets. The cap member may be formed from one sheet of compressed decellularized porcine liver. These sheets can be dried flat for anywhere from 5 hours to 7 days at either room temperature or in an oven at temperatures up to 100° C., for example. Alternatively, lyophilizers can be used to dry the material.

The suture may be a braided absorbable suture of about 60 cm in length (about 30 cm once folded) with an approximate absorption profile of: 75% of original tensile strength maintained at 2 weeks, 50% of original tensile strength maintained at 4 weeks, and complete resorption of the material in about 6 months, for example.

The tamp member and handle member, in one embodiment, may be formed of plastic. A range of dimensions for both parts include but is not limited to a tamp member length of about 4 cm to about 10 cm, e.g., about 6 cm to about 9 cm, about 6 cm to about 8 cm, about 7 cm to about 9 cm or about 7 cm to about 8 cm and a handle member length of about 2 cm to about 7 cm, e.g., about 3 cm to 4 cm are 4 to 5 cm. An exemplary outer diameter for both components is about 2 mm to 3 mm, 3 mm to 4 mm or 4 mm to 5 mm, e.g., or a diameter that is less than the diameter of the compressed tunnel filler.

The tunnel brush may have a range of dimensions, e.g., an overall length that is about 30 cm to 80 cm with an outer diameter of about 1 mm to 2 mm. In one embodiment, a stiff brush portion has a length of about 2 cm and about 0.5 cm diameter is centered in the overall length. A loop of suture may be incorporated on each end of the tunnel brush. The integrated loop functions like a noose and can be adjusted to any length. The additional loop length provides enough material (e.g., about 20 cm) to interface with the tunnel brush.

In one embodiment, the tunnel filler may be added to the looped suture first by threading the loose ends through the filler. In another embodiment, the suture threaded through the tunnel filler in one direction and then back through the filler to form the loop. The tamp member and handle member are subsequently added to the suture.

The product may be sterilized, for example by electron beam (E-beam) irradiation, e.g., at 25 kGy to 35 kGy.

When introducing the device into a tunnel wound, the user can brace the handle member piece through its connection to the suture while pushing the tamp member and tunnel filler toward the tunnel wound tract. The tamp member and handle member are added last.

Once the device is in place, the parallel lines of suture may be cut, in one embodiment, between the tunnel filler or cap member (if present) and the tamp member. This will leave two free suture ends available to tie a simple knot together at the end of the tunnel filler material.

All publications, patents and patent applications are incorporated herein in their entirety by reference.

The above Detailed Description includes references to the accompanying drawings, which form a part of the Detailed Description. The Detailed Description should be read with reference to the drawings. The drawings show, by way of illustration, one embodiment in which the present devices and methods can be practiced.

The above Detailed Description is intended to be illustrative and not restrictive. For example, the above-described embodiments, also known as examples, or one or more features or components thereof can be used in combination with each other. Also, various features or components have been grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, a device for treatment of a tunnel wound comprises a suture, a tunnel filler, and a cap member. The suture is folded to form a proximal loop and two distal ends. The tunnel filler is compressed onto the suture between the proximal loop and the two distal ends. And the cap member is positioned on the suture distal to the tunnel filler.

In Example 2, the device of Example 1 is optionally configured such that the tunnel filler comprises decellularized mammalian ECM.

In Example 3, the device of Example 2 is optionally configured such that the decellularized mammalian ECM comprises decellularized porcine ECM.

In Example 4, the device of Example 2 is optionally configured such that the decellularized mammalian ECM comprises decellularized liver ECM.

In Example 5, the device of Example 2 is optionally configured such that decellularized mammalian ECM is cross-linked.

In Example 6, the device of any one or any combination of Examples 1-5 is optionally configured such that the tunnel filler is compressed onto the suture between the proximal loop and the two distal ends, and the proximal loop extends beyond a proximal end of the tunnel filler.

In Example 7, the device of Example 6 is optionally configured such that the tunnel filler is radially compressed onto the suture.

In Example 8, the device of Example 6 is optionally configured such that the tunnel filler is torsionally compressed onto the suture.

In Example 9, the device of Example 6 is optionally configured such that the proximal end of the tunnel filler has a tapered shape or a conical shape.

In Example 10, the device of any one or any combination of Examples 1-9 is optionally configured such that the suture passes through a center of the cap member, and the cap member is sized and shaped to hold the tunnel filler in place within the tunnel wound.

In Example 11, the device of any one or any combination of Examples 1-10 is optionally configured such that the cap member comprises decellularized mammalian ECM, polycaprolactone, polylactic acid (PLA), polyglycolic acid (PGA), or co-polymers of PLA and PGA.

In Example 12, the device of any one or any combination of Examples 1-11 is optionally configured such that the cap member is in the form of a compressed sheet.

In Example 13, the device of any one or any combination of Examples 1-12 optionally further comprises a tamp member positioned over the suture distal to the cap member.

In Example 14, the device of Example 13 optionally further comprises a handle member attached to the suture and positioned distal to the tamp member.

In Example 15, the device of any one or any combination of Examples 1-14 is optionally configured such that a folded length of the suture is about 20 centimeters to about 40 centimeters, inclusive.

In Example 16, the device of any one or any combination of Examples 1-15 is optionally configured such that a compressed diameter of the tunnel filler is about 1 millimeter to about 6 millimeters, inclusive.

In Example 17, the device of any one or any combination of Examples 1-16 is optionally configured such that a pre-compressed size of the tunnel filler is about 3 centimeters to about 8 centimeters in both width and height.

In Example 18, the device of any one or any combination of Examples 1-17 is optionally configured such that a compressed length of the tunnel filler is about 2 centimeters to about 12 centimeters, inclusive.

In Example 19, the device of any one or any combination of Examples 1-18 is optionally configured such that the suture comprises a material that is resorbable post-implantation.

In Example 20, a kit comprises the device of any one or any combination of Examples 1-19 and a tunnel brush. The tunnel brush is configured to engage with the proximal loop of the suture.

In Example 21, a device for treatment of a tunnel wound comprises a resorbable suture and a tunnel filler. The resorbable suture has a proximal end and a distal end. The tunnel filler comprises decellularized mammalian ECM and is compressed onto the suture between its proximal end and the distal end. In Example 22, the device of Examples 21 further comprises a cap member positioned on the suture distal to the tunnel filler.

In Example 23, the device of Example 22 further comprises a tamp member positioned over the suture distal to the cap member.

In Example 24, the device of any one or any combination of Examples 21-23 is optionally configured such that a proximal end of the tunnel filler has a tapered shape or a conical shape.

In Example 25, the device of any one or any combination of Examples 21-24 is optionally configured such that the proximal end or the distal end of the suture forms a loop.

In Example 26, a method of treating a tunnel wound in a mammal comprises: introducing a device, including a suture and a tunnel filler compressed onto the suture, into the tunnel wound; applying pressure to an end of the tunnel filler using a tamp member slidably positioned along the suture; removing the tamp member from the suture; and securing an end of the suture to an end of the tunnel wound.

In Example 27, the method of Example 26 is optionally configured such that treating the tunnel wound includes treating an anal fistula.

In Example 28, the method of Example 27 is optionally configured such that securing the end of the suture to the end of the tunnel wound includes securing a proximal end loop of the suture to an anal side opening of the anal fistula.

In Example 29, the method of any one or any combination of Examples 26-28 is optionally configured such that introducing the device into the tunnel wound includes removing a tunnel brush, which is engaged with an end loop of the suture, from the tunnel wound.

In Example 30, the method of any one or any combination of Examples 26-29 is optionally configured such that applying pressure to the end of the tunnel filler includes applying pressure to a cap member positioned on the suture distal to the end of the tunnel filler.

In Example 31, the method of Example 30 optionally further comprises removing excess tunnel filler and positioning the cap member against the remaining tunnel filler within the tunnel wound.

In Example 32, the method of Example 30 is optionally configured such that securing the end of the suture to the end of the tunnel wound includes securing a knot against a surface of the cap member.

In Example 33, the method of any one or any combination of Examples 26 and 29-31 is optionally configured such that securing the end of the suture to the end of the tunnel wound includes securing a first end of the suture to a first end of the tunnel wound and securing a second end of the suture to a second end of the tunnel wound.

In Example 34, the method of any one or any combination of Examples 26-33 optionally further comprises hydrating the tunnel filler, thereby expanding the tunnel filler within the tunnel wound.

In Example 35, the method of Example 34 is optionally configured such that the tunnel filler expands about two-fold from its compressed, non-hydrated size.

In Example 36, the device or method of any one or any combination of Examples 1-35 can optionally be configured such that all components or options recited are available to use or select from.

Certain terms are used throughout this patent document to refer to features or components. As one skilled in the art will appreciate, different people may refer to the same feature or component by different names. This patent document does not intend to distinguish between components or features that differ in name but not in function.

For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” refers to a range of numbers that one of skill in the art considers equivalent to the recited value (e.g., having the same or similar function or result). The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “patient” is intended to include mammals, such as for human or veterinary applications. The terms “distal” and “proximal” are used to refer to a position or direction relative to an operating physician. “Distal” and “distally” refer to a position that is distant from, or in a direction away from, the physician. “Proximal” and “proximally” refer to a position that is near, or in a direction toward, the physician.

The scope of the present devices and methods should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1-35. (canceled)

36. A device for treatment of a tunnel wound, comprising:
a suture folded to form a proximal loop and two distal ends; and
a tunnel filler positioned on the suture between the proximal loop and the two distal ends.

37. The device of claim 36, wherein the tunnel filler comprises decellularized mammalian extracellular matrix (ECM).

38. The device of claim 37, wherein the decellularized mammalian ECM is decellularized porcine ECM or decellularized liver ECM.

39. The device of claim 36, wherein the tunnel filler is compressed onto the suture between the proximal loop and the two distal ends, such that the proximal loop extends proximally of a proximal end of the tunnel filler.

40. The device of claim 39, wherein the tunnel filler is radially compressed or torsionally compressed onto the suture.

41. The device of claim 40, wherein a compressed diameter of the tunnel filler is about 1 millimeter to about 6 millimeters, inclusive.

42. The device of claim 40, wherein a pre-compressed size of the tunnel filler is about 3 centimeters to about 8 centimeters, inclusive, in both width and height.

43. The device of claim 40, wherein a compressed length of the tunnel filler is about 2 centimeters to about 12 centimeters, inclusive.

44. The device of claim 36, wherein the proximal end of the tunnel filler has a tapered shape or a conical shape.

45. The device of claim 36, further comprising a cap member positioned on the suture distal to the tunnel filler.

46. The device of claim 45, wherein the suture passes through a center of the cap member, and wherein the cap member is sized and shaped to hold the tunnel filler in place within the tunnel wound.

47. The device of claim 45, wherein the cap member comprises decellularized mammalian ECM, polycaprolactone, polylactic acid (PLA), polyglycolic acid (PGA), or co-polymers of PLA and PGA.

48. The device of claim 45, wherein the cap member is in the form of a compressed sheet.

49. The device of claim 45, further comprising a tamp member positioned over the suture distal to the cap member.

50. The device of claim 49, further comprising a handle member attached to the suture and positioned distal to the tamp member.

51. The device of claim 36, wherein a folded length of the suture is about 20 centimeters to about 40 centimeters, inclusive.

52. The device of claim 36, wherein the suture comprises a material that is resorbable post-implantation.

53. A method of treating an anal fistula in a mammal, comprising:
introducing a device, including a suture and a tunnel filler compressed onto the suture, into the anal fistula;
applying pressure to an end of the tunnel filler using a tamp member slidably positioned along the suture;
removing the tamp member from the suture; and
securing a proximal end loop of the suture to an anal side opening of the anal fistula.

54. The method of claim 53, wherein introducing the device into the anal fistula includes removing a tunnel brush, which is engaged with the proximal end loop of the suture, from the tunnel filler.

55. The method of claim 53, further comprising hydrating the tunnel filler, thereby expanding the tunnel filler within the anal fistula about two-fold from its compressed, non-hydrated size.
